SEQUENCE LISTING (8 sequences)

SEQ ID NO: 1 - DNA, 216 bp, JC virus
  CDS (1)..(216)
atggttcttcgccagctgtcacgtaaggcttctgtgaaagttagtaaaacctggagtggaactaaaaaaagagctcaaaggattttaatttttttgttagaatttttgctggacttttgcacaggtgaagacagtgtagacgggaaaaaaagacagagacacagtggtttgactgagcagacatacagtgctttgcctgaaccaaaagctacatag

SEQ ID NO: 2 - protein, 71 aa, JC virus
MVLRQLSRKASVKVSKTWSGTKKRAQRILIFLLEFLLDFCTGEDSVDGKKRQRHSGLTEQTYSALPEPKAT

SEQ ID NO: 3 - DNA, 20 bp, Artificial
  DNA Primer
ggtgccaacctatggaacag

SEQ ID NO: 4 - DNA, 20 bp, Artificial
  DNA Primer
agtctttagggtcttctacc

SEQ ID NO: 5 - DNA, 21 bp, Artificial
  DNA Primer
tgtgcactctaatgggcaagc

SEQ ID NO: 6 - DNA, 21 bp, Artificial
  DNA Primer
ctaggtacgccttgtgctctg

SEQ ID NO: 7 - DNA, 20 bp, Artificial
  DNA Primer
atggttcttcgccagctgtc

SEQ ID NO: 8 - DNA, 21 bp, Artificial
  DNA Primer
ctatgtagcttttggttcagg

(19) United States
(12) Patent Application Publication
(10) Pub. No.: US 2004/0259767 A1
(43) Pub. Date: Dec. 23, 2004

(54) Treatment of pml targeting jc virus agno

(76) Inventors: Kazuo Nagashima, Hokkaido (JP); Hirofumi Sawa, Hokkaido (JP); Yuki Okada, Hokkaido (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/496,391

(22) PCT Filed: Jun. 5, 2002
(86) PCT No.: PCT/JP02/05528

(30) Foreign Application Priority Data
Nov. 22, 2001 (JP) 2001356836

(51) Int. Cl. A61K 39/395

(52) U.S. Cl. 514/2; 424/130.1

(57) ABSTRACT

It is intended to provide an effective method of treatment of PML (human progressive multifocal leukoencephalopathy). Inhibition of JC virus proliferation in JC virus-infected cells by suppression of JC virus agnogene; and a method of inhibition of the canceration of JC virus-infected cells.

### FIELD OF THE INVENTION

The present invention relates to a method of treatment of progressive multifocal leukoencephalopathy (PML) by inhibition of JC virus agno, and a pharmaceutical composition thereof.

### BACKGROUND OF THE INVENTION

PML is a demyelinating disease caused by the infection with a JC virus (JCV). JCV is a double-stranded circular DNA virus belonging to polyomavirus family, and latently infects the urinary tract system of 70% or more of normal healthy individuals. In immunocompromized circumstance, JCV replicates in the central nervous system, infects oligodendroglia, proliferates, and results in lethal demyelination of brain.

### SUMMARY OF THE INVENTION

Previously, PML was a relatively rare disease, however; it becomes more popular along with pandemic of an acquired immunodeficiency syndrome (AIDS) and immunosuppressive therapy after transplantation of bone marrow and other organs. Especially in such a transplantation, it cannot be avoided that a patient is subjected to immunosuppressive condition. Overcoming of PML is very important in success of implantation therapy. Although cytosine arabinoside (Ara-C), a DNA synthesis inhibitor, and interferon β have been employed in the treatment of PML, effective therapies against PML are not established yet.

### BRIEF DESCRIPTION OF THE DRAWINGS

FIG. 1 shows the results of the test in which both a mutant JC viral DNA (Δagno) in which an initiation codon, ²⁷⁷ATG (Met) of agnoprotein, was converted into ²⁷⁷CGA (Arg) and a wild-type viral DNA (WT) expressing agnoprotein were introduced into IMR-32. After various periods from transfection, total RNAs were isolated from the cells, and subjected to an RT-PCR assay, following the 2% TAE agarose gel electrophoresis to compare the mRNA levels between the cells transfected by Δagno or WT viruses. The “M” indicates a 100-bp ladder DNA marker, numbers indicate periods (days) aftertransfection of DNAs, LT indicates large-T antigen.

### BEST MODE FOR CARRYING OUT THE INVENTION

The inventors had been investigated the glia tissue-specific tropism of JCV infection and tried to establish an effective treatment of PML. Analysis of JCV-encoded agnogene whose function is unknown revealed that JCV having a variation in the agno gene lacking agnoprotein was significantly suppressed expression as described in EXAMPLES. Thus, the present inventors established the invention.

An artificial control of the expression of an agno gene by using a gene engineering technology including application of antisense oligonucleotides or RNAi, or an inhibition of the agnoprotein by using an anti-agnoprotein antibody can suppress JCV infection, contributing to development of PML treatment.

Accordingly, the invention relates to the following aspects:

1. A method for suppression of JCV infection in JCV infected cells by inhibition of JCV agnogene.

2. A method for suppression of canceration caused by JCV infection by inhibition of JCV agnogene.

3. A method for treatment of progressive multifocal leukoencephalopathy by inhibition of JCV agnogene.

4. A method for treatment of human progressive multifocal leukoencephalopathy by inhibition of JCV agnogene.

5. The method according to any one of the above-mentioned 1 to 4 introducing an antisense oligonucleotide against JCV agnogene into JCV-infected cells by inhibition of JCV agnogene.

6. The method according to any one of the above-mentioned 1 to 4 introducing an RNA fragment complementary with JCV agnogene into JCV-infected cells by inhibition of JCV agnogene.

7. A method for suppression of JCV proliferation in JCV infected cells by treatment with anti-agnoprotein antibody to suppress agnoprotein.

8. A method for suppression of canceration of JCV infected cells by treatment with anti-agnoprotein antibody to suppress agnoprotein.

9. A method for treatment of progressive multifocal leukoencephalopathy by treatment with anti-agnoprotein antibody to suppress agnoprotein.

10. A method for treatment of human progressive multifocal leukoencephalopathy by treatment with anti-agnoprotein antibody to suppress agnoprotein.

11. The method according to any one of the above-mentioned 7 to 10 wherein the anti-agnoprotein antibody is a polyclonal antibody.

12. The method according to any one of the above-mentioned 7 to 10 wherein the anti-agnoprotein antibody is a monoclonal antibody.

13. An inhibitor of the expression of JCV agnogene comprising of an antisense oligonucleotide of JCV agnogene.

14. A pharmaceutical composition comprising as an active ingredient an antisense oligonucleotide of JCV agnogene.

15. A pharmaceutical composition for treatment of human progressive multifocal leukoencephalopathy comprising as an active ingredient an antisense oligonucleotide of JCV agnogene.

16. An inhibitor of expression of JCV agnogene comprising of an RNA fragment complementary with JCV agnogene.

17. A pharmaceutical composition comprising as an active ingredient an RNA fragment complementary with JCV agnogene.

18. A pharmaceutical composition for treatment of a human progressive multifocal leukoencephalopathy comprising as an active ingredient an RNA fragment complementary with JCV agnogene.

19. A suppressant of the proliferation of JCV consisting of an anti-agnoprotein antibody.

20. The suppressant of the proliferation according to the above-mentioned 19 wherein the anti-agnoprotein antibody is a polyclonal antibody.

21. The suppressant of the proliferation according to the above-mentioned 19 wherein the anti-agnoprotein antibody is a monoclonal antibody.

22. A suppressant of the canceration of JCV-infected cells consisting of an anti-agnoprotein antibody.

23. The suppressant of the canceration according to the above-mentioned 22 wherein the anti-agnoprotein antibody is a polyclonal antibody.

24. The suppressant of the canceration according to the above-mentioned 22 wherein the anti-agnoprotein antibody is a monoclonal antibody.

25. A pharmaceutical composition comprising as an active ingredient an anti-agno protein antibody.

26. A pharmaceutical composition for treatment of human progressive multifocal leukoencephalopathy comprising as an active ingredient an anti-agnoprotein antibody.

27. The pharmaceutical composition according to the above-mentioned 25 or 26 wherein the anti-agnoprotein antibody is a polyclonal antibody.

28. The pharmaceutical composition according to the above-mentioned 25 or 26 wherein the anti-agnoprotein antibody is a monoclonal antibody.

The JCV infected cells in each method according to the invention include the cells derived from humans and non-human animals, for example, mammalian animals. In addition, the target of the inventive treatment is progressive multifocal leukoencephalopathy, especially a human progressive multifocal leukoencephalopathy.

In a method of the invention, by transfection either with an antisense oligonucleotide directed to JCV agnogene or with an RNA fragment complementary with agnogene into JCV infected cells followed by the complementary binding of these transduced antisense oligonucleotide or RNA fragment to an mRNA transcribed from JCV agnogene whereby inhibition of agnogene, alternatively, introduction of an anti-agnoprotein antibody into JCV-infected cells, it becomes possible to suppress the proliferation of JCV in JCV-infected cells, to suppress canceration in JCV-infected cells, and also to treat progressive multifocal leukoencephalopathy.

Such an antisense oligonucleotide, RNA fragment or anti-agnoprotein antibody can be introduced by any method known to those skilled in the art. For example, the introduction into the JCV-infected cells can be accomplished by a lipofection, electroporation and other methods. Especially, when using a capsid protein of JCV as a vector, the antisense oligonucleotide can efficiently be introduced into the target site. The timing and the level of the introduction of the antisense oligonucleotide, RNA fragment or anti-agnoprotein antibody into JCV-infected cells may vary depending on the sex, age, body weight and condition of the patient to be treated, and may be appropriately selected.

The invention also relates to inhibitors of expression of JCV agnogene including an antisense oligonucleotide of JCV agnogene or an RNA fragment complementary with JCV agnogene, and a pharmaceutical composition comprising the same as an active ingredient, especially a pharmaceutical composition for treatment of human progressive multifocal leukoencephalopathy. By using such inhibitors or pharmaceutical composition, methods of the invention described above can be practical.

An antisense oligonucleotide directed to JCV agnogene or an RNA fragment complementary with agnogene can be prepared readily by chemical synthesis or PCR reaction and other methods known to those skilled in the art based on the nucleotide sequence represented by SEQ ID No.1. Such an antisense oligonucleotide may not necessarily be the full-length nucleotide sequence of JCV agnogene represented by SEQ ID No.1, and may have nucleotide sequence with shorter length. While the number of the bases possessed by the RNA fragment complementary with agnogene is not limited particularly, and the one which has 15 to 30 nucleotides, preferably about 25 nucleotides, and which is complementary with the 5′-terminal region of JCV agnogene represented by SEQ ID No.1 may be exemplified.

The invention furthermore relates to a suppressant of the proliferation of JCV consisting of an anti-agnoprotein antibody and a suppressant of the canceration of JCV-infected cells, as well as a pharmaceutical composition comprising as an active ingredient an anti-agnoprotein antibody especially for treatment of human progressive multifocal leukoencephalopathy. The anti-agnoprotein antibody may be a polyclonal or monoclonal antibody, which may readily be prepared using a technology known in the art. For example, JCV agnoprotein is injected as an immunogen to an animal such as mouse or rat whereby preparing a polyclonal anti-agnoprotein antibody. Alternatively, a cell fusion technology employing a spleen cell of such an immunized animal can be utilized to prepare the monoclonal antibody. In addition, a gene recombination technology may also be employed to produce various chimera antibodies which cause no rejection upon administration to a human, such as a humanized antibody in which the region except for an antigen determinant or variable region has been replaced with human region. Any of such agents or pharmaceutical compositions can be employed to conduct a method of the invention described above. The timing and the level of the introduction into JCV-infected cells may appropriately be selected by those skilled in the art depending on the sex, age, body weight and condition of the patient to be treated.

Any of the various agents and pharmaceutical compositions described above may contain any optional pharmaceutically acceptable components well known in the art such as various auxiliary agents, formulatory aids, as well as other active ingredients, and may be in any dosage form such as a solid, solution, emulsion, gel, sol, powder, granule and the like. The amounts of the active ingredient and other components, ratios, consumption levels, doses and treatment intervals may appropriately be selected by those skilled in the art depending on the sex, age, body weight and condition of the patient to be treated.

### EXAMPLES

The invention is further described in the following EXAMPLES, however it is not limited by these EXAMPLES.

### Example 1

### Method for Preparation of Agnogene Mutant JCV

Agnogene-Containing Virus Gene Subcloning and Nucleoide Substitution by Site-Directed Mutagenesis Method:

JCV, a 5,130-bp double-stranded circular DNA virus, in which agnogene is encoded at nt.277-492 from the numbers represent the nucleotide the replication initiation site as the 1st nucleotide (nt.1), and in which agnoprotein consisting of 71 amino acids is transcribed and translated. The present inventors made an attempt, using site-directed mutagenesis method, to prepare a viral gene without agnogene transcription products. From a plasmid pJC1-4→pJCV (HSRRB, VGO 15) in which the full-length JCV gene of the Mad-1 type as a prototype of JCV reported by Frisque et al in 1984 (Journal of Virology, 51: p.458-469, 1984, “Human polyomavirus JC virus genome”) had been inserted, an about 1 kbp gene containing agnogene was isolated by digestion with Hind III and Apa I, and subcloned into pBluescript SK+ (Stratagene). Using this plasmid as a template together with Unique Site Elimination mutagenesis kit (Amersham Pharmacia Biotech) [1], agnoprotein initiation codon ²⁷⁷ATG (Met) was converted into ²⁷⁷CGA (Arg), or [2] ²⁸⁰GTT (Val) immediately after agnoprotein initiation codon is converted into ²⁸⁰TAA (Stop), whereby producing a viral gene which could not express agnoprotein. This variation-containing virus gene was also isolated by digestion with Hind III and Apa I, and inserted into the plasmid pJC1-4→pJCV, to obtain a full-length JCV variant agnogene.

Intracellular Introduction of Viral Gene:

JCV can be artificially produced by introducing a full-length JCV genome into human neuroblastoma-derived cell line, IMR-32. The inventors cleaved the agno-deficient viral gene [1] and [2] out of the vector sequence by Bam HI to isolate a 5,130 bp JCV gene. Thereafter, the viral DNA was introduced into IMR-32 cells using a gene transfection reagent, Effectene (Qiagen). Since replication of JCV is known to be relatively slow, the serum concentration of the cell culture was reduced from 10 to 5% on the day following to transfection to reduce cell proliferation, whereby increasing virus replication.

### Example 2

Viral RNA Recovery and Investigation of JCV Replication by Reverse Transcription-Polymerase Chain Reaction (RT-PCR) Method:

Following 10 minutes (day 0), 3 days, 5 days and 7 days after transfection of JCV DNA, total RNA was isolated from the cells using a total RNA extraction reagent, ISOGEN (NIPPON GENE). One μg of total RNA was treated with DNase I to eliminate viral DNA, thereafter RNA was transcribed into cDNA using ™First-strand synthesis system for RT-PCR (GIBCO, BRL). The cDNA was used as a template for the following 3 primer sets in PCRassays.

| Primers No.1 (for T antigen) | | |
|---|---|---|
| Tag-F: | 5′-ggtgccaacctatggaacag-3′ | (nt. 4427-4408, 20mer) |
| Tag-R: | 5′-agtctttagggtcttctacc-3′ | (nt. 4255-4274, 20mer) |
| Primers No.2 (for VP1) | | |
| VP1-F: | 5′-tgtgcactctaatgggcaagc-3′ | (nt. 1828-1848, 21mer) |
| VP1-R: | 5′-ctaggtacgccttgtgctctg-3′ | (nt. 2039-2019, 21mer) |
| Primers No.3 (for agnogene) | | |
| agno-F: | 5′-atggttcttcgccagctgtc-3′ | (nt. 277-296, 20mer) |
| agno-F: | 5′-ctatgtagcttttggttcagg-3′ | (nt. 492-472, 21mer) |

The PCR conditions are as follows.

When using Primers No.1 (for T antigen):

94° C. for 1 minute→(94° C. for 30 seconds; 58° C. for 30 seconds; 72° C. for 30 seconds)×3 cycles→(94° C. for 30 seconds; 60° C. for 30 seconds; 72° C. for 30 seconds)×25 cycles→72° C. for 10 minutes

When using Primers No.2 (for VP1):

94° C. for 1 minute→(94° C. for 30 seconds; 63.5° C. for 30 seconds; 72° C. for 30 seconds)×28 cycles→72° C. for 10 minutes

When using Primers No.3 (for agnogene):

94° C. for 1 minute→(94° C. for 30 seconds; 48° C. for 30 seconds; 72° C. for 30 seconds)×3 cycles→(94° C. for 30 seconds; 53° C. for 30 seconds; 72° C. for 30 seconds)×25 cycles→72° C. for 10 minutes

An equal amount of each PCR product thus amplified under the condition specified above was subjected to an electrophoresis on a 2% TAE agarose gel, and mRNA levels of the cells transfected with either Δagno or WT viral DNA. In order to confirm of absence of viral DNA in the resultant PCR product, total RNA which had not been subjected to reverse transcription reaction was also used as a template for the PCR. As a positive control for the PCR, a pEGFP-N1 vector (Clontech) was transfeccted at the same time with the viral DNA, and the EGFP gene was simultaneously amplified with PCR. In addition, as a positive internal control for total RNA extraction and reverse transcription reaction, human β-actin gene was also amplified. For positive control for the PCR reaction, cDNA from JCV producing cell lines, JCI was applied, and for a negative control, deionized water (DW) was used.

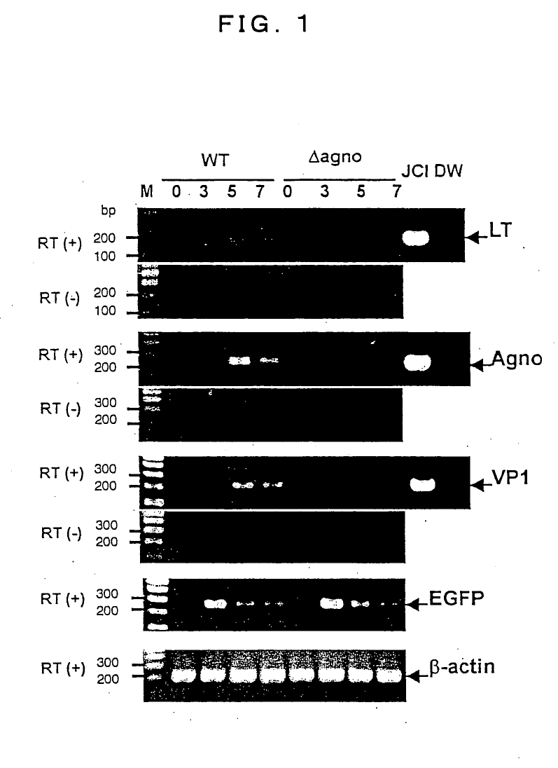

The results of the RT-PCR described above are shown in FIG. 1. Since we have obtained the same result using mutants [1] and [2], the result of the mutant [1] was represented as Δagno. Wild-type viral DNA (WT) exhibited expression of both of early protein, Large T antigen and late proteins, agnoprotein and VP1 mRNAs at 3 to 5 days after the transfection. On the contrary, mRNAs of Large-T antigen were almost not detectable, in mutant virus (Δagno), and those of VP1 were just at an only detectable level on the day 7 after transfection.

Industrial Applicability

Agnogene is encoded in the most upstream in the late protein transcription region of JCV and translates a protein consisting of 71 amino acids (8 kDa). The function of agnoprotein is still unknown. In general, mutation of late protein does not have an effect on expression of early proteins. The above-mentioned experiment employing the agno mutant virus synthesized by the inventors revealed that deletion of agnoprotein caused marked inhibition or suppression of expression of JCV early protein, Large-T antigen.

Large-T antigen has an ability to induce canceration of cells via binding with various cellular proteins and DNAs in the host cell, and is also essential as an initiation factor of replication and a transcription factor for the proliferation process of JCV itself. Accordingly, the invention was proven to be capable of inhibiting expression of agnoprotein whereby inhibiting function of a potent JCV-activating factor, Large-T antigen, resulting in a significant suppression of viral proliferation.

Based on these results, a method of the invention for suppressing the proliferation of JCV by allowing an oligonucleotide having a sequence complementary with JCV agnogene (antisense oligonucleotide) to exert its effect whereby inhibiting (suppressing) the agnogene expression (antisense method) is provided as an effective PML treatment as a substitute for conventional therapeutic methods.

An antisense oligonucleotide can readily be designed when the target gene sequence is known, and can be synthesized in a large amount at a low cost within a short period. It is also biologically advantageous, since it is highly unlikely for a resistant virus to appear differently from a chemotherapy, and also it has a little side effect on a host cell. An antisense method has already been applied as a therapeutic approach against a persistent infectious disease such as AIDS or cancer in several clinical trials, and the antisense therapy against a cytomegaloviral retinal infection in AIDS patients has already been brought into a practical use.

In the invention, the size of the base sequence of a JC virus agnogene targeted in PML treatment is as small as 216 bp, which is short enough to select appropriate regions for synthsis of antisense nucleotides, and the variation of the virus gene is less frequent compared with a RNA virus such as HIV.

1. A method for suppression of JCV infection in JCV infected cells by inhibition of JCV agnogene.

2. A method for suppression of canceration caused by JCV infection by inhibition of JCV agnogene.

3. A method for treatment of progressive multifocal leukoencephalopathy by inhibition of JCV agnogene.

4. A method for treatment of human progressive multifocal leukoencephalopathy by inhibition of JCV agnogene.

5. The method according to any one of claims 1 to 4 introducing an antisense oligonucleotide against JCV agnogene into JCV-infected cells by inhibition of JCV agnogene.

6. The method according to any one of claims 1 to 4 introducing an RNA fragment complementary with JCV agnogene into JCV-infected cells by inhibition of JCV agnogene.

7. A method for suppression of JCV proliferation in JCV infected cells by treatment with anti-agnoprotein antibody to suppress agnoprotein.

8. A method for suppression of canceration of JCV infected cells by treatment with anti-agnoprotein antibody to suppress agnoprotein.

9. A method for treatment of progressive multifocal leukoencephalopathy by treatment with anti-agnoprotein antibody to suppress agnoprotein.

10. A method for treatment of human progressive multifocal leukoencephalopathy by treatment with anti-agnoprotein antibody to suppress agnoprotein.

11. The method according to any one of claims 7 to 10 wherein the anti-agnoprotein antibody is a polyclonal antibody.

12. The method according to any one of claims 7 to 10 wherein the anti-agnoprotein antibody is a monoclonal antibody.

13. An inhibitor of the expression of JCV agnogene comprising of an antisense oligonucleotide of JCV agnogene.

14. A pharmaceutical composition comprising as an active ingredient an antisense oligonucleotide of JCV agnogene.

15. A pharmaceutical composition for treatment of progressive multifocal leukoencephalopathy comprising as an active ingredient an antisense oligonucleotide of JCV agnogene.

16. An inhibitor of expression of JCV agnogene by an RNA fragment complementary with JCV agnogene.

17. A pharmaceutical composition comprising as an active ingredient an RNA fragment complementary with JCV agnogene.

18. A pharmaceutical composition for treatment of human progressive multifocal leukoencephalopathy comprising as an active ingredient an RNA fragment complementary with JCV agnogene.

19. A suppressant of the proliferation of JCV consisting of an anti-agnoprotein antibody.

20. The suppressant of the proliferation according to claims 19 wherein the anti-agnoprotein antibody is a polyclonal antibody.

21. The suppressant of the proliferation according to claims 19 wherein the anti-agnoprotein antibody is a monoclonal antibody.

22. A suppressant of the canceration of JCV-infected cells consisting of an anti-agnoprotein antibody.

23. The suppressant of the canceration according to claims 22 wherein the anti-agnoprotein antibody is a polyclonal antibody.

24. The suppressant of the canceration according to claims 22 wherein the anti-agnoprotein antibody is a monoclonal antibody.

25. A pharmaceutical composition comprising as an active ingredient an anti-agno protein antibody.

26. A pharmaceutical composition for treatment of human progressive multifocal leukoencephalopathy comprising as an active ingredient an anti-agnoprotein antibody.

27. The pharmaceutical composition according to claims 25 or 26 wherein the anti-agnoprotein antibody is a polyclonal antibody.

28. The pharmaceutical composition according to claims 25 or 26 wherein the anti-agnoprotein antibody is a monoclonal antibody.
